(19) United States
(12) Patent Application Publication
(10) Pub. No.: US 2011/0020859 A1
(43) Pub. Date: Jan. 27, 2011

(54) DEVICE COMPRISING A COLOURED AND BIODEGRADABLE POLYMER LAYER FOR ANALYZING THE AGE AND/OR QUALITY OF A NATURAL PRODUCT (INTEGRATED FRESHNESS INDICATOR)

(75) Inventors: Maria Bauer, Salzburg (AT); Fritz Pittner, Wien (AT); Georg Bauer, Eferding (AT)

Correspondence Address: K&L Gates LLP, P.O. Box 1135, CHICAGO, IL, 60690, US

(73) Assignees: UNIVERSITÄT WIEN, Wien (AT); MAX F. PERUTZ LABORATORIES GMBH, Wien (AT)

(21) Appl. No.: 12/528,281

(22) PCT Filed: Dec. 13, 2007
(86) PCT No.: PCT/EP2007/063896

§ 371 Date: Oct. 4, 2010

(30) Foreign Application Priority Data
Mar. 1, 2007 (AT) A 327/2007

(51) Int. Cl. G01N 21/75; C12Q 1/00; B05D 1/18; B05D 5/00; G01N 33/02; C12Q 1/04

(52) U.S. Cl. 435/34; 422/425; 435/4; 427/430.1; 427/256; 436/164; 436/20; 977/755

(57) ABSTRACT

The present invention relates to the field of analyzing the age and/or quality of certain natural products, for example foods. The invention also relates to devices for analyzing said age and/or quality as well as to methods for preparing such devices, to methods for analyzing natural products and to their use.

### FIELD OF THE INVENTION

The present invention relates to the field of optical sensors for analyzing the age and/or quality of a natural product comprising foods and cosmetic products.

In this context, the invention describes a sensor, in which an analyte-sensitive layer together with a dye using a cross-linking agent is applied onto a carrier as thin layer assembly. The invention also describes the preparation and use of the sensor.

### BACKGROUND OF THE INVENTION

Control of the quality of perishable foods and/or cosmetic products is a critical task throughout the storage, production, distribution and consumption/use of such natural products.

Many foods are subject to spoilage, which may be caused by improper production and/or handling and/or storage. If, for example, perishable products such as milk or meat are exposed to excessive temperatures during transport, they will age and spoil prematurely.

Aging processes leading to the spoilage of foods are often caused by microorganisms. Examples for the spoilage of foods caused by microorganisms comprise: the spoilage of fish and meat are caused by the infestation with spoilage-causative organisms such as Bacillus sp., Clostridia, Proteus sp., Areomonas sp., Acinetobacter, Serratia, Flavobacterium, Actinomyces, Mould sp. and so on; the spoilage of milk and milk products are caused by the infestation with decay-bacteria (proteolytes) such as for example Cocci, Enterobakteriaceae, aerobic spores-building organisms, Pseudomonas sp., mould and Lactic acid bacteria, as well as the spoilage of foods containing carbohydrates such as bread caused by molds.

At present, the spoilage of such foods is detected by consumers by means of tasting such foods or by the visible decay of foods in the advanced stages of decay. The main disadvantage of the technical solutions known today is that the spoilage of meat is not visible for the consumer, although the consumer may suffer major health damage by the consumption. For this reason, foods are nowadays randomly tested in laboratories. In general, these tests rely on the proliferation of the microorganisms after several days and the visible detection of the extracted and multiplied DNA. Both methods of detection are time consuming and are not available to the consumer.

From the literature is known U.S. Pat. No. 6,625,479 in the name of Weber and Stanley: an implantable sensor which provides an optical signal which can be seen or measured, wherein the sensor is biologically degradable. In contrast to this, the present application describes a sensor wherein the sensing layer itself is degraded and particularly because of the coloration of this layer the degradation can be made visible; the degradation in turn is proportional to the freshness of the tested food. U.S. Pat. No. 6,975,245 also describes a biodegradable sensor but without the degradation itself being the signal. WO 2005110207 also describes a sensor which is surrounded by a covering of a biologically degradable polymer, but again without the degradation of this polymer being the actual sensor signal. Furthermore, application JP 2004344056 by Ito et al. is known from the literature wherein the activity of microorganisms is detected in that a nutrient and a dye are added to a biodegradable polymer and applied in thicknesses of the layer ranging from 1 to 15 micrometers and the degradation thereby can be made visible within 28 days. In the present application, a substantially thinner layer (100 to 1000 nanometers) of a biodegradable polymer together with a dye and preferably a cross-linking agent is applied. Thereby, the sensitivity is increased in a favourable way by more than one order of magnitude and the reaction time of the sensor is reduced from several days to few hours (both at 4° C.). Furthermore, the addition of nutrients is not necessary. Thereby the sensor is suited to measure the spoilage of fresh foods in real-time integrated into a packaging and not, as proposed in the application by Ito et al., to measure fermented foods and waste of foods.

Concerning the previous application of the co-inventors Bauer and Pittner, the present application is not dependent on the use of a 3 layer setup wherein a cross-linked degradable polymer layer is applied between a minor and a metallic island layer. In the present application, a homogenous layer comprising a polymer, preferably a cross-linking agent and a dye may be applied onto a carrier.

Apart from DNA, bacteria also contain specific enzymes. The relatively low activity of the enzymes secreted by bacteria to digest substrates has thus far prevented the use of specific protein molecules for the detection of bacteria. The concentration of such enzymes correlates with the amount of bacteria present. Naturally occurring lytic enzymes within the cells contribute also to the aging of fresh foods after their release from the inside of the cell (inter alia, meat mellows upon release of such enzymes). Thus, the detection of enzymatic activity by the consumer may be an essential contribution to the increase in confidence of consumers to a product and in monitoring conditions of delivery and storage.

To ease quality control of foods for consumers, there is a need for a device and/or sensor that indicates the condition of foods in a cheap and easily visible way. The device and/or sensor is used for the detection of specific reactions of degradation that occur during the aging of foods. Such aging processes are often associated with the spoilage of foods. The quality of the product may, therefore, be checked before consumption even without any knowledge of transport and storage conditions. The device and/or sensor should give information about aging process and the microbial contamination during the total period of storage life.

### OBJECTS AND SUMMARY OF THE INVENTION

It is an objective of the present invention to provide a device which can be used to analyze the age and/or quality of natural products, e.g. foods.

It is yet another objective of the present invention to provide a method for preparing such a device.

It is a further objective of the present invention to provide a method for analyzing the age and/or quality of a natural product with said device.

It is an objective of the present invention to describe the use of said device for the analysis of the age and/or quality of a natural product.

It is thus an object of the present invention to describe a sensor which makes the specific activity of microbial enzymes as well as of degrading enzymes originating from meat which are responsible for the decay of foods, visible to the consumer of foods.

These and other objectives of the present invention, as they will become apparent from the ensuing description, are solved by the subject matter of the independent claims. The dependent claims relate to some of the preferred embodiments of the invention.

According to one aspect of the invention, a device for analyzing the age and/or quality of a natural product is provided comprising a coloured and biodegradable polymer layer positioned on a carrier layer. Said device is configured in such a way that biomolecules capable of degrading said polymer layer are allowed to contact said polymer layer. Furthermore, said device is configured in such a way that a degradation of said polymer layer results in a colour change visible to the human eye.

In a preferred embodiment of the present invention, the carrier layer has a thickness of 10 nm to 500 μm. In a further preferred embodiment of the present invention, said coloured and biodegradable polymer layer has a thickness of 50 to 5000 nm.

In a further preferred embodiment of the invention, the carrier layer is selected from the group of carriers comprising polyethylenterephtalate, polyamide, glass, polyethylene, polycarbonate, polypropylene, silicone, and ceramics. In yet another embodiment of the present invention, said carrier layer is coloured. In a preferred embodiment of the present invention, said coloured carrier layer is made of polyethylenterephtalate and has a thickness of 20 to 100 μm.

In another preferred embodiment of the invention, biomolecules capable of degrading said polymer layer are allowed to penetrate said carrier layer in order to contact said polymer layer.

In a preferred embodiment of the present invention, the coloured and biodegradable polymer layer is degradable by biomolecules comprising enzymes and/or catabolic metabolites. In a further preferred embodiment of the present invention, said biodegradable polymer layer is selected from the group of polymers comprising PLA, PLGA, PHB, and polyvinylcaprolactame.

In another preferred embodiment of the invention, the coloured and biodegradable polymer layer is coloured using a dye and/or pigment selected from the group comprising food colouring, acryl-dyes, azo-dyes, fluorescence-dyes and luminescence-dyes.

In another preferred embodiment of the present invention, the coloured and biodegradable polymer layer has a thickness of 100 to 1000 nm.

In still another embodiment of the present invention, said coloured and biodegradable polymer layer additionally comprises a cross-linking agent to obtain a certain degree of cross-linking in said polymer layer wherein said cross-linking agent is selected from the group of bifunctional radical cross-linking agents.

In yet a further aspect of the present invention, the device also comprises a reference device.

In yet another aspect of the present invention, a method for preparing such a device is provided. This method comprises the steps of
(a) providing a carrier layer
(b) applying a coloured and biodegradable polymer layer onto said carrier layer.

In yet a further aspect of the methods of the present invention, in step (a) of the method mentioned above a carrier layer is provided which is coloured in a different colour than said polymer layer.

In yet another aspect of the methods of the present invention, in step (b) of the method mentioned above the polymer layer has been coloured by mixing it with a dye and/or pigment before applying it onto said carrier layer.

In yet another aspect of the methods of the present invention, the polymer layer is applied by dip coating or film printing.

The present invention relates in one aspect to a method for analyzing the age and/or quality of a natural product comprising foods and cosmetic products. This method comprises the following steps:
(a) providing a device as described above
(b) contacting said device with a natural product
(c) determining the colour of said device
(d) comparing the colour of said device to the colour of a reference device
(e) determining the age and/or quality of said natural product according to this comparison.

The afore described method for analyzing the age and/or quality of a natural product comprises in a further embodiment a step (step b) listed above) wherein the polymer layer of said device is being contacted in step b) directly with said natural product.

The afore described method for analyzing the age and/or quality of a natural product comprises in another embodiment a step (step b) listed above) wherein the carrier layer of said device is being contacted in step b) with said natural product in such a way that biomolecules are allowed to penetrate the carrier layer and contact the polymer layer.

In a preferred embodiment, a device as described above is used for the analysis of the age and/or quality of a natural product comprising foods and cosmetical products.

The present invention relates in a further preferred embodiment to the use of a device as described above for the analysis of the age and/or quality of a natural product by detecting microorganisms present in the natural product.

The present invention relates in a further preferred embodiment to the use of a device as described above for the analysis of the age and/or quality of a natural product by detecting enzymes and/or catabolic metabolites of microorganisms and/or of the natural product via the degradation of said biodegradable polymer by said enzymes and/or catabolic metabolites.

The present invention also relates in a further preferred embodiment to the use of a device as described above for the analysis of the age and/or quality of a natural product wherein the degree of cross-linking of the polymer layer is proportional to the kinetics of degradation of said polymer layer by enzymes and/or catabolic metabolites.

### DESCRIPTION OF THE FIGURES

FIG. 1: Schematic view of a sensor platelet with integrated reference field. The figure shows a schematic cross section of a sensor platelet with the following layers:
1: carrier layer
2: biodegradable coloured polymer layer
3: transparent and/or biodegradable polymer layer

FIG. 2: Picture of a sensor platelet which has been incubated with different concentrations of meat juice. This figure shows a picture of a sensor platelet, which has been incubated with different concentrations of enzyme and old meat juice as well as buffer:
4: old meat juice
5: enzyme
6: buffer
7: sensor platelet

FIG. 3: Schematic view of the packaging with integrated multianalyte array. The figure shows a schematic view of a packaging with integrated freshness indicator according to the invention.
8: packaging with partly opened lid
9: sensor field in the form of a stripe
10: reference field

FIG. 4: Exemplary use of a device according to the invention to analyze the age and/or quality of meat. In the left case, both, the device according to the invention and the reference device, are shown. The device according to the invention is formed as stripe in the left picture and as square in the right picture. The reference device comprises three colours indicating the quality of the product ranging from “ok” to “harmful”. The patterning of the device depicted here does not show a certain embodiment, it is rather meant to illustrate that the device may have different colours, e.g. red, yellow, blue or white. Also, the carrier layer of the device may be transparent resulting in a transparent device upon degradation of the coloured and biodegradable polymer layer.

### DETAILED DESCRIPTION OF THE INVENTION

As has been set out above, there is a need for a device which allows the analysis of the age and/or quality of a natural product by the consumer.

The present invention provides devices and methods for solving this need. While describing in detail exemplary embodiments of the present invention, definitions important for understanding the present invention are also given.

As used in this specification and in the appended claims, the singular forms of “a” and “an” also include the respective plurals unless the context clearly dictates otherwise.

In the context of the present invention, the terms “about” and “approximately” denote an interval of accuracy that a person skilled in the art will understand to still ensure the technical effect of the feature in question. The term typically indicates a deviation from the indicated numerical value of ±10% and preferably ±5%.

It is to be understood that the term “comprising” is not limiting. For the purposes of the present invention the term “consisting of” is considered to be a preferred embodiment of the term “comprising of”. If hereinafter a group is defined to comprise at least a certain number of embodiments, this is meant to also encompass a group which preferably consists of these embodiments only.

The term “natural product” in the context of the present invention comprises any product which is subjected to spoilage and/or decay and, therefore, possesses a certain time frame in which it may be used according to its purpose. As set out in the background section, using an e.g. spoiled natural product such as eating spoiled food may lead to major health problems. Foods form a very large class of such natural products. This class is comprised of fish, meat, milk products, vegetables, carbohydrate-containing foods such as bread, and the like. Another class of natural products comprises cosmetic products, which are also subjected to spoilage and/or decay, e.g. in case of inadequate storage and/or delivery conditions and aging processes. The term “natural product” in the context of this invention does not define a “natural product” in a way that it has to be untreated. The natural product may be treated or untreated. Any natural product according to the invention may be treated and/or e.g. used in preparation processes, such as e.g. cooking, baking, boiling, freezing, and the like. “Natural” in the context of the invention rather implies that the product, although it may be pretreated, is still a substrate for spoilage and/or decay processes by e.g. microorganisms. The natural product may also be packaged in any way known to the person skilled in the art.

The term “age and/or quality” relates to the natural product as defined above. As mentioned before, natural products possess different time frames, in which they may be used according to their purposes. A very important aspect is the age of the product because of the correlation of contamination by e.g. microorganisms and time. The longer the incubation time, the more concentrated and severe the contamination. In case of inadequate storage and/or delivery conditions, this correlation may change and favour an even more severe contamination within a shorter time. For this reason, the natural product should always be subjected to controls even if it is not incubated for a long time. Therefore, “age” and “quality” in the context of the present invention mainly refer e.g. in case of foods to the edibility (“freshness”) of such foods.

The term “biomolecules” in the context of the present invention defines molecules present in or secreted from the natural product to be analysed and/or present in or secreted from any further object associated with said natural product, e.g. a microorganism. Therefore, the biomolecule may derive from e.g. a food such as meat or from a microorganism associated with said meat. Preferably, such biomolecules comprise enzymes, such as phospholipases, hydrolases, pronases, proteinases (such as proteinase K), esterases of different types, lipases and the like. Such enzymes are capable of degrading the polymer layer as described below. Biomolecules according to the present invention also comprise any molecules present in or secreted from the natural product to be analysed and/or present in or secreted from any further object associated with said natural product, e.g. a microorganism, of non-enzymatic origin which are capable of degrading the biodegradable polymer layer, i.e. for example intermediates of the metabolism of such microorganisms associated with said natural product. Such non-enzymatic molecules are defined in the context of the invention as catabolic metabolites. Examples for such catabolic metabolites comprise volatile acids, volatile bases, volatile aldehydes, volatile mercaptans and sulfur compounds. Common to both (to the enzymes as well as to the catabolic metabolites) is their ability to degrade the polymer layer of the device described herein.

According to the invention, a coloured and biodegradable polymer layer is positioned on a carrier layer.

The term “coloured” defines that the colour of the polymer layer is visible to the human eye preferably under normal conditions such as day light. The colour may comprise any colour visible to the human eye, such as for example white, blue, green, red. As defined below in more detail, the carrier layer is either coloured itself (but in this case in a different colour than the polymer layer and, therefore, a colour change occurs upon degradation of the polymer layer) or is transparent such that it is possible to determine a degradation of said polymer layer by the whole device becoming transparent, i.e. for example that the natural product and the colour of said natural product, respectively, is visible to the consumer when looking at the device. In any case, the polymer layer is not transparent, but always displays a colour and, therefore, a degradation always results in a colour change and/or a change to transparency of the whole device.

The biodegradable polymer layer as defined below may be coloured by a dye such as a dye or pigment selected from the group comprising food colourings such as E120, E122, E123, E124, E127, E140, E142, acryl-dyes, azo-dyes, fluorescence-dyes and luminescent dyes. Of course, said dyes or pigments may be combined in any possible combination, for example to obtain a specific colour of said polymer layer. Also, the polymer material as described below may itself be already coloured. The intensity of the colour may be dependent on the thickness of said polymer layer. Therefore, the degree of degradation may be proportional to the intensity of the colour of said polymer layer. Thus, an analysis over a range is possible and the device may not only result in a binary signal.

The polymer layer of the present invention is made of a polymer. A “polymer” may in general be classified as either being a naturally occurring polymer (e.g. as present in or secreted by microorganisms such as agarose in algae) or a “man-made”, synthetic polymer. A naturally-occurring polymer may also be referred to as “biopolymer”. Thus, a synthetic polymer according to the definition used here cannot be found in nature in exactly the same condition and/or modification and/or conformation. However, every naturally occurring polymer (biopolymer) which has been subjected to modification (such as e.g. cross-linking) resulting in conditions and/or conformations which are not naturally occurring has been transformed into a synthetic polymer and is thus no biopolymer any more by consequence. Thus, a modified biopolymer can be classified as synthetic polymer if its state of modification is not found in nature. Preferably, polymers used in the present invention and described in further detail below belong to the class of synthetic polymers as defined above.

The term “biodegradable” defines that the polymer layer of the present invention is degradable by biomolecules comprising enzymes and/or catabolic metabolites as defined above. Therefore, e.g. enzymes/catabolic metabolites secreted by microorganisms present in foods or enzymes/catabolic metabolites secreted by the natural product itself are capable of degrading said polymer layer.

The material of the polymer layer is preferably chosen from the group comprising polylactic acid (PLA), poly-L-lactic acid (PLLA), PLGA, PHB and Polyvinylcaprolactame (PVCL) or any other polymer, which falls under the classification of a polymer degradable by biomolecules as defined above. This may also comprise gelatine, agarose, dextrose, lipids, cellulose, starch, chitin, polyhydroxyalkanoates, poly(-caprolactone) (PCL) or PCL-systems, poly(ethylene/butylene succinate) or poly(ethylene/butylene adipate). Furthermore, the polymer layer may not comprise any further nutrients and/or reactive chemicals and/or any further materials or compounds, but may only be comprised of said biodegradable polymer layer, a dye, a cross-linking agent and a solvent. In case the material of said polymer layer is degraded by enzymes and/or catabolic metabolites, this is accompanied by a change in the colour of the polymer layer as the degradation also leads to a loss of the colour of said polymer layer. As set out above, the thickness of the layer may be proportional to the intensity of the colour of said polymer layer. Ultimately, the degradation leads to a change of the colour of the whole device.

In another preferred embodiment of the invention, the biodegradable polymer layer additionally comprises a cross-linking agent. This cross-linking agent may be a bifunctional agent, such as e.g. diisocyanat, glutardialdehyde or Desmodur (Desmodur 2460 M, Bayer). Desmodur products based on diphenylmethane diisocyanate (MDI), toluene diisocyanate (TDI), hexamethylene diisocyanate (HDI) and isophorone diisocyanate (IPDI) may also be used. For the setup of the system it needs to be understood that the degree of cross-linking of the biodegradable polymer layer correlates with its degradation time. The higher the degree of cross-linking of the biodegradable polymer layer, the longer it takes the biomolecules described above to degrade said layer. By adjusting the degree of cross-linking of the biodegradable polymer layer, the sensitivity over time of the device may thus be adjusted.

In still another preferred embodiment of the invention, the biodegradable polymer layer additionally comprises a solvent. This solvent may be selected from the group comprising chloroform, chloroform +50% v/v EtAc, toluol and trifluoroethanol or 2-butanone.

In a preferred embodiment of the invention, the biodegradable polymer layer has a thickness of about 75 to about 4500 nm. In one of the more preferred embodiments of the invention, the polymer layer has a thickness of about 150 to about 3000 nm. In a further preferred embodiment, the polymer layer has a thickness of about 300 nm. Preferably, the scale of the thickness of the polymer layer is in the nanometric scale. For the setup of the system it needs to be understood that the thickness of the biodegradable polymer layer correlates with its degradation time. The thicker the biodegradable polymer layer, the longer it takes the biomolecules described above to degrade said layer. By adjusting the thickness of the biodegradable polymer layer, the sensitivity over time of the device may thus be adjusted.

Thus, in one embodiment of the invention, the sensitivity of the device is mainly regulated by adjusting the degree of cross-linking of the polymer layer as well as its thickness and by choosing a certain material for the biodegradable polymer layer as listed above.

In a preferred embodiment of the invention, PLA may be used as polymer in a concentration of about 16.5% (weight/volume) with Desmodur as cross-linking agent in a concentration of about 2% (volume/volume) together with the food colouring E140 in a concentration of 8% (w/v) for the coloured and biodegradable polymer layer. Said layer may have a thickness of about 350 nm and a green colour due to the food colouring E 140. As solvent, trifluorethanole may be used for said layer.

In yet another preferred embodiment of the invention, PLA may be used in a concentration of about 25% (w/v) together with the food colouring E 140 in a concentration of about 10% (w/v) for the coloured and biodegradable polymer layer. Said layer may have a thickness of about 400 nm and a green colour due to the food colouring E 140. As solvent, 2-butanone may be used for said layer.

In an also preferred embodiment of the invention, PLA may be used as polymer in a concentration of about 10% (weight/volume) with Desmodur as cross-linking agent in a concentration of about 0.001% (volume/volume) together with the food colouring E122 in a concentration of 15% (w/v) for the coloured and biodegradable polymer layer. Said layer may have a thickness of about 300 nm and a red colour due to the food colouring E 122. As solvent, trifluorethanole may be used for said layer.

The preferred embodiments of the coloured and biodegradable polymer layer mentioned above may all be positioned onto a carrier layer as defined in detail below made of PET with a thickness of 50 μm. Said PET layer may be transparent.

The term “carrier layer” describes a layer onto which the degradable polymer layer is positioned. Preferably, the material used for the carrier layer is inert to any reactions with the natural product. In a certain embodiment, the carrier layer may be of such material and configured such that biomolecules as defined above are not able to penetrate said layer. In such an embodiment the polymer layer as defined above needs to be either directly or via a permeable second carrier layer (which is positioned on the opposite side of the first carrier layer) in contact with said natural product in order to contact biomolecules as defined above. The carrier layer may in another embodiment be configured such that biomolecules as defined above are able to penetrate said layer in order to contact the polymer layer on the other side of the carrier layer. However, it needs to be understood that the carrier layer itself is not degradable by biomolecules as set out above. Furthermore, the carrier layer should be stabile in biological buffers. In certain embodiments, the carrier layer is used to fix the device in the packaging of the natural product. In other preferred embodiments, the carrier layer is useful for the production of the device as set out below. The carrier layer may e.g. be in a preferred embodiment a PET-film. In another embodiment, the carrier layer may e.g. be any standard transparent film, on top of which the polymer layer is positioned. The carrier layer may be part of the preparation-process and may in one embodiment be of the same origin as the packaging material for the natural product. In other preferred embodiment, the additional carrier layer is an inorganic or organic carrier selected from the group comprising polyethylenterephtalate, polyamide, glass, polyethylene, polycarbonate, polypropylene, silicone, and ceramics. In case the carrier layer displays a colour, this colour is different than the colour of the biodegradable polymer layer. Upon degradation of the polymer layer, the carrier layer itself becomes visible and thus the colour of the carrier layer becomes exposed and visible to the consumer. As mentioned below, in case the polymer layer is green and the carrier layer is red, the colour of the device changes from green to red upon degradation of the polymer layer, which might be interpreted as intuitive signal by the consumer for a negative change in the quality of the natural product.

As for the biodegradable polymer layer, the carrier layer may be coloured by a dye such as a dye or pigment selected from the group comprising food colouring, acryl-dyes, azo-dyes, fluorescence-dyes and luminescent dyes. Of course, said dyes or pigments may be combined in any possible combination, for example to obtain a specific colour of said carrier layer. Also, the material of said carrier as described above may itself be already coloured.

The permeable carrier layer may be about 20 μm to about 100 μm thick, preferably it may be about 40 μm to about 80 μm thick, more preferably it may be about 50 μm to about 70 μm thick. In case the carrier layer is non-permeable for biomolecules as defined above, it may be about 5 nm to about 500 nm thick, preferably it may be about 50 nm to about 250 nm thick, more preferably it may be about 100 nm to about 150 nm thick.

The whole setup of the invention is configured such that a degradation of the polymer layer leads to a change of the colour of the device which is visible to the human eye. The expression “whole setup” comprises for this reason all layers of the device, their material, the thickness of each layer and so on. However, the coloured biodegradable polymer layer may be the only layer which is degradable. Apart from said coloured biodegradable polymer layer, there are no other reactive chemicals present which could indicate the presence of reactive stimuli by changing the colour due to e.g. chemical reactions such as pH-changes, hydrations, oxidations and so on. The colour change of the device described in the present invention may thus be solely due to the degradation of the coloured polymer layer resulting in a loss of said colour.

The thicknesses as well as the colours mentioned in the context of the carrier layer and the material of the polymer layer itself thus reflect certain embodiments of the invention. They can also be in a different range as long as the setup works, wherein the degradation of the coloured polymer layer leads to a change of the colour of the device which is visible to the human eye.

In still another preferred embodiment of the invention, the device comprises a second transparent carrier layer which is positioned on the polymer layer but on the opposite side of the first carrier layer described above. This may be a layer having protective properties. The second carrier layer may be a non-degradable transparent carrier layer, e.g. a non-degradable transparent polymer layer. Such a second carrier layer may be a transparent hydrogel layer. This hydrogel layer me be positioned on the polymer layer. In one embodiment, this layer may be in direct contact with the natural product and allow the penetration of biomolecules to the polymer layer. The hydrogel layer may act as protection layer for the device and may represent a diffusion layer. The hydrogel layer may preferably be a crosslinked and/or stabilized food-contact compatible polymer layer which is swelling in contact with water and ensures that water is attracted in proximity to the biodegradable layer. The crosslinked hydrogel layer may in a preferred embodiment be a poly-acrylic-acid (PAA) layer. The PAA may have been neutralized with KOH so that it does not change the pH in the microenvironment of the biodegradable layer. In mentioned embodiments, this second carrier layer may preferably be transparent and is thus not interfering with the setup mentioned above, wherein the degradation of the polymer layer leads to a change in the colour of the device.

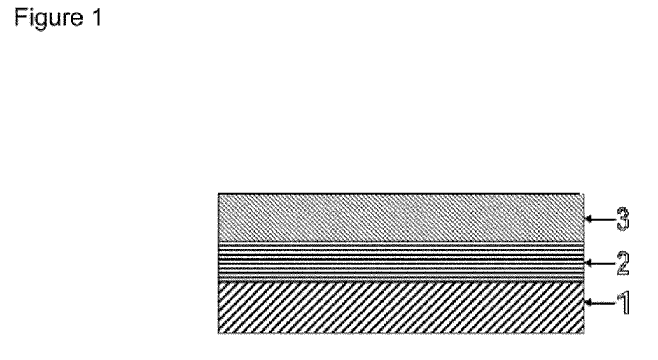

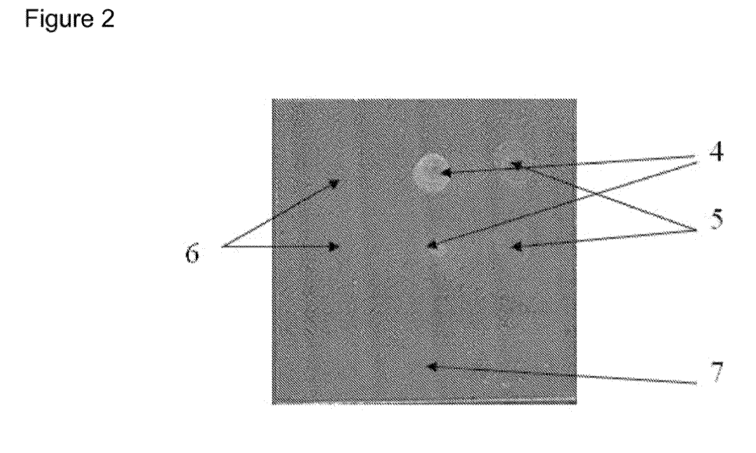

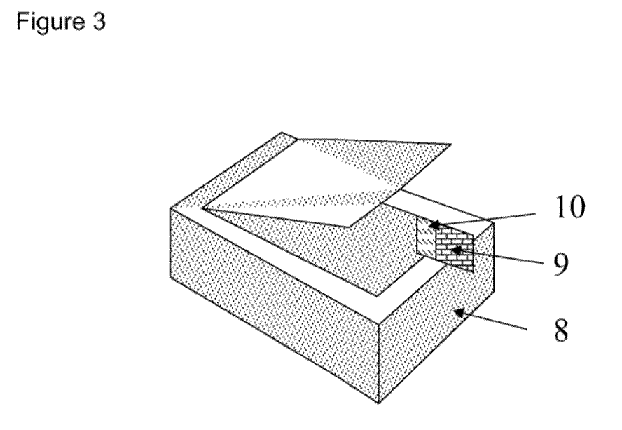

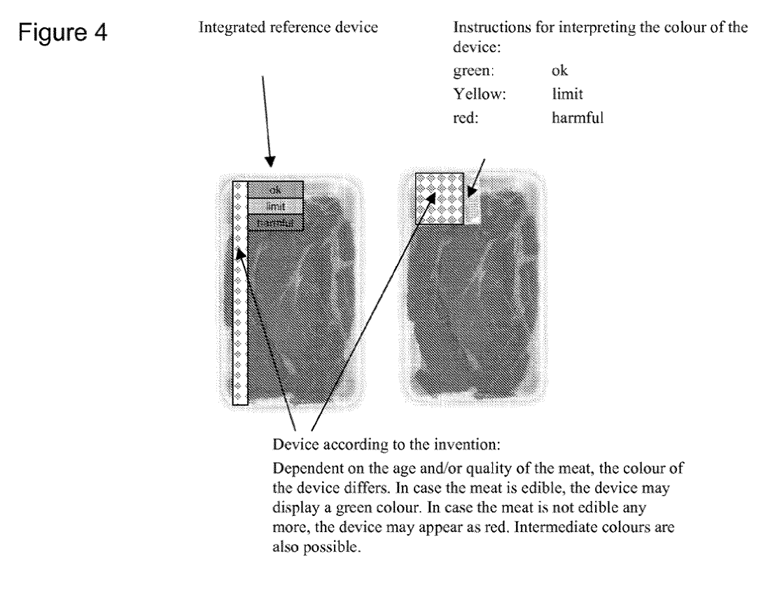

The device according to the invention may have different forms. In a preferred embodiment of the invention, the device may have the form of a square (FIG. 4). In a further preferred embodiment, the device may be a stripe. Both devices may be integrated into the packaging of e.g. meat (see FIG. 4). Both forms may have an identical surface area, but according to their form they cover different specific areas of e.g. meat. In case of the square, a certain area of meat is covered to a very good extent, whereas in case of a stripe, a broad area of meat is covered to a very good extent. In both cases, this is meant to account for the possibility that the spoilage of meat is not necessarily a homogenous process and, therefore, may start at different areas and at different points of time. By having the form of a stripe reaching from one end of the packaging to the other end or as big square on one spot (see FIG. 4), the inventors try to account for this problem by covering different areas of meat. In other embodiments of the invention, the form of the device may be a circle, rectangle, ellipse or any other suitable form.

Furthermore, the device according to the invention may be comprised of a combination of at least two devices and thus cover a broad range of reactivity and sensitivity due to the characteristics of each single device. An example for such a device is the following preferred embodiment wherein coloured biodegradable polymer layers with different cross-linking properties are placed next to each other in such a way, that a range of defined biodegradable polymer layers is formed, e.g. a polymer layer with a cross-linking range of 4.5%, next to 8.0%, next to 12.0%, next to 20.0%. Also, a gradient might be formed ranging from 4.5% to 20% cross-linking.

The different biodegradable polymer layers (the gradient, respectively) may be positioned onto a single carrier layer. In such a setup, each biodegradable polymer layer shows a different reaction kinetic with the biomolecules defined above. In case the cross-linking range is low (for example 4.5%), the polymer layer is destroyed fast associated with an early optical signal, i.e. a colour change in this region. If the cross-linking range is high (for example 20%), the polymer layer is destroyed much later due to higher resistance to enzymatic and/or chemical reactions and the colour change, therefore, occurs later in time in the respective region. With a device built in this setup, it is possible to cover different time points/frames of the reaction of biomolecules as defined above and with the biodegradable polymer layer and, ultimately, different stages of the decay/spoilage are monitored.

Furthermore, another example for such a combined device is the following preferred embodiment wherein differently coloured biodegradable polymer layers, each of which exhibiting a specificity for certain enzymes (obtained e.g. by different polymer materials), are placed next to each other combined with references in such a way, that one can deduce which enzymes are present due to a selective colour change in an area of the device. Again, the different biodegradable polymer layers may be positioned onto a single carrier layer. In such a setup, specific enzymes and, therefore, for example specific microorganisms secreting unique enzymes may be determined. Also, it is possible to use such a setup to detect specific enzymes of certain foods: one part of such a device only reacts to certain enzymes secreted by fish by changing the colour, whereas another part of the device only reacts to certain enzymes secreted by meat by changing the colour. Of course, by using such a setup it is also possible to distinguish between certain species of meat and their enzymes, respectively. Clearly, such a device has a very broad area of application due to its broad reactivity over a wide range of foods.

The term “reference device” according to the present invention defines a device of a specific colour or a colour range, wherein the colour/colour range is not subjected to a change of colour. To this aim, the reference device is in a preferred embodiment of the invention coloured by any technique known to the person skilled in the art. In another embodiment of the invention, the reference device comprises a coloured polymer layer which is not degradable by biomolecules as mentioned above and therefore does not change its colour in case it is contacted by said biomolecules. Accordingly, the colour of the reference device after exposure to such biomolecules does not change. Thus, the reference device may have one colour which is identical to the colour of the device according to the invention in case the biodegradable polymer layer is totally intact. Furthermore, the reference device may have a second colour, which is identical to the device according to the invention in case the biodegradable polymer layer is substantially up to totally degraded by biomolecules as mentioned above. In this embodiment, the consumer is able to compare the colour of the device according to the invention to two possible colour-conditions of the polymer layer of the invention. Of course, the reference device may comprise more than one or two colours for comparison reasons. In an also preferred embodiment, the reference device does not display certain specific colours, but is comprised of a coloured non-degradable polymer layer ranging from the thickness of the device of the invention before any degradation to zero and, therefore, displays a colour range. Again, the consumer may compare the colour of the device according to this invention to said colour range. The reference device may be positioned directly next to the device of the invention. Furthermore, the reference device may be inert and, therefore, may not influence the natural product itself.

Furthermore, in other embodiments of the present invention, methods for preparing the aforementioned devices are disclosed. In one embodiment, a carrier layer selected from the carriers mentioned above (e.g. glass or polyamide) is provided. Onto said carrier layer, a coloured biodegradable polymer layer, e.g. green PLA, is applied. In another preferred embodiment, a further layer, namely a second transparent non-degradable carrier layer (e.g. a hydrogel layer) is applied onto said polymer layer.

As mentioned above, in a preferred embodiment of the invention, the carrier layer is coloured in a different colour than the polymer layer before said layer is provided. This may be for example a red colour. In such an embodiment, it is important to note that the two colours, the colour of the carrier layer and the colour of the polymer layer, are different such that they cannot be confused by the consumer. If for example the carrier layer is red, the polymer layer may be green.

In an also preferred embodiment, the biodegradable polymer layer has been coloured by mixing it with a dye as mentioned above before applying it onto the carrier layer. Said mixing step therefore precedes the application step and may be done in any way known to the skilled person in the art.

In preferred methods for preparing such devices, the polymer layer may be applied by dip coating or film-printing techniques, such as gravure printing, or by spin coating. Such techniques are routine methods to the skilled person in the art. Any other technique known to the person skilled in the art leading to the application of thin polymer layers onto other layers may also be used. In preferred embodiments, PLA is used as material for the polymer layer. In the methods for preparing the polymer layer, PLA may be used in a concentration (weight/volume) ranging from about 1,5% w/v to about 25% w/v in a suitable solvent, such as chloroform, trifluorethanole, Toluole, 2-butanone and the like, wherein trifluorethanole is preferred. The concentration of the cross-linking agent used is also critical for the method of preparing the polymer layer. Desmodur might be used as cross-linking agent in a concentration (volume/volume) ranging from about 0.001% v/v to about 5.0% v/v. In a preferred aspect of the invention, the polymer layer is prepared with about 16.5% (w/v) PLA in triflouroethanole and about 0.001% (v/v) Desmodur as cross-linking agent by gravure printing. According to the colouring step mentioned above, the layer may also comprise any dye responsible for colouring which has been added and mixed into the solution.

The sensor is prepared according to the invention in that a nanometric layer of a biodegradable polymer, as for example poly-lactic-acid (PLA), is applied onto a carrier. The biodegradable layer consists of a degradable polymer, a cross-linking agent and a dye. The colour disappears upon the degradation of the sensor layer. A reference area consisting of a layer which is non-degradable under the conditions used and which has been coloured by the same dyes or pigments can be printed next to the degradable coloured layer wherein the reference does not change upon contact with enzymes. Therefore, the consumer finds a reference while analyzing the sensor. FIG. 1 shows a schematic sensor. Also, a sensor can be optimized in its use for the consumer in that the coloured biodegradable polymer is printed onto a coloured carrier. While degradation occurs, the colour of the carrier becomes visible. Thereby a colour change from green to red can be achieved for the human eye and an intuitive interpretation of the signal is assured.

In still other embodiments of the invention, methods for analyzing the age and/or quality of a natural product are provided. In one aspect of the invention, the natural product is directly contacted with the device such that the following set up of layers is present:
carrier layer, e.g. glass
polymer layer, e.g. PHB
optionally a second carrier layer, e.g. a hydrogel

The second carrier layer contacts the natural product, e.g. meat. This second carrier layer is, as set out above, permeable for biomolecules and transparent. In this setup, the biomolecules as defined above are able to penetrate the hydrogel layer and contact the polymer layer. Alternatively, if no second carrier layer is present, the biomolecules directly contact said polymer layer. In order to analyze the colour of the device, the consumer may open the packaging to an extent to which the device is visible to the consumer, i.e. by separating the natural product from the device in order to look at the polymer layer of the device. Again, the carrier layer may be part of the packaging of the natural product or may be a separate carrier, such as a PET-film. In this setup, the carrier layer can be of any material as there is no need for the carrier layer to be transparent or permeable for biomolecules. Therefore, in this direct setup, the consumer is able to analyze the colour of said device by partly opening up the packaging and looking at the device which may be integrated into the packaging of the natural product via said carrier layer.

In yet another embodiment of the invention, further methods for analyzing the age and/or quality of a natural product are provided. In one aspect of the invention, the natural product is directly contacted with the device such that the following set up of layers is present:
carrier layer, e.g. PET film
polymer layer, e.g. PLA
optionally a second carrier layer, e.g. a hydrogel

Here, the carrier layer contacts the natural product, e.g. meat. In this setup, the carrier layer used must be permeable for biomolecules. In this setup, the biomolecules as defined above are able to penetrate the carrier layer and contact the polymer layer. The consumer is looking onto the polymer layer and, therefore, can directly see the colour of the device if the packaging is transparent. Therefore, in this indirect setup, the consumer is able to analyze the colour of said device by directly looking at the device which may be integrated into the packaging of the natural product.

The method for analyzing the age and/or quality of a natural product may comprise the comparison of the colour of the device to the colour of a reference device as defined above. The reference device may have one, two or several fixed colours corresponding to possible colours of the device of the invention according to possible degradation conditions of the polymer layer as already set out above. In case the polymer layer is totally degraded, the device might be e.g. of white colour instead of a e.g. blue colour in case the polymer layer is not affected at all. Of course, the colour of the carrier layer is white in this example. The reference device may in this case be comprised of a white and a blue colour and, therefore, the consumer is able to compare the colour of the device to the reference-device and determine the age and/or quality of a natural product. If the device according to the present invention corresponds to the white colour of said reference device, the natural product is not intact any more and should not be used according to its purpose. On the other hand, if the device according to the invention corresponds to the blue colour of said reference device, the age and/or quality of said natural product is a condition, in which the natural product may be used according to its purpose. As set out before the reference device may display a colour range. In this case, the consumer can compare the colour of the device according to the invention to the colour range of the reference device and determine the age and/or quality of the natural product accordingly.

In other embodiments, the present invention is concerned with the use of a device according to the present invention for the analysis of the age and/or quality of a natural product. Via the degradation of a biodegradable polymer layer by enzymes and/or catabolic metabolites of the natural product itself or any associated organism, said age and/or quality can be analyzed. The higher the concentration of said biomolecules, the thinner the polymer layer of said device. Also, the degree of cross-linking of the polymer layer is an important parameter of the kinetics of degradation. Thus, the higher the degree of cross-linking, the slower the degradation of said polymer layer. This, in turn, decreases the loss in thickness of the polymer layer. Overall, there is a correlation between the presence of e.g. enzymes of microorganisms responsible for spoiling foods and the thickness of the polymer layer. The thickness in turn correlates with the colour of the device visible to the human eye. Therefore, ultimately, the presence of e.g. microorganisms responsible for spoiling foods correlates with the colour of said device. Thus, the device may be used for analyzing the age and/or quality of a natural product. In case foods are analyzed, the edibility of such foods may be analyzed by the consumer.

The sensor may thus be used for the detection of specific reactions of degradation that occur during the aging of e.g. foods or cosmetic products. Such aging processes are often associated with e.g. the spoilage of foods.

Also, the device according to the invention is thus used in a preferred embodiment to analyze if the cold chain of foods has been handled correctly. As set out above, most of the microorganisms responsible for the spoilage of foods preferably proliferate at 37° C. Both, the activity of microorganisms as well as the release and activity of cell-based lytic enzymes are temperature-dependent according to Arhenius. Therefore, many foods, e.g. meat, are stored and transported at temperatures below 37° C., preferably at 4° C. or even frozen at −20° C. to maintain an unfavourable temperature range for such microorganisms. As transport includes different storage areas maintained at such low temperatures as e.g. cold storage houses or adequate transport vehicles, the whole delivery process from the place of production to the place of offering such foods (e.g. a supermarket) is referred to as the cold chain. Therefore, the device according to the invention may be used by the consumer of the natural product to analyze if the natural product indeed has been handled according to the cold chain. Alternatively, the person involved in presenting and selling the product (e.g. an employee of a supermarket) may check at the arrival the quality of the natural product to decide whether storage and/or transport have been handled according to the cold chain and, therefore, whether the natural product may be presented to the consumer.

Surprisingly, it has been found that a biologically degradable polymer in an optical thin layer setup is translating the activity of enzymes into visible signals. The decay of the thin layers leads to defined changes in the colour of the surface. From the literature, such underlying degradation conditions are known, but could thus far only be visualized in the laboratory. While there is ultimately no guarantee on this theory, one assumes based on the present data that the enzymes penetrate into the optical thin layer setup and attack the bonds between the polymer-units. It has been observed that the stability of the polymer layer gets reduced and the layer brakes down or disintegrates and thereby the cohesion of the optical thin layer systems breaks down. Thereby the analysis of the freshness of meat is possible in real-time.

Further preferred embodiments:
1. Sensor characterized in that a 50 to 500 nm thick, preferably 100 to 1000 nm thick, biodegradable and coloured, cross-linked polymer layer is applied onto a carrier in such a way that enzymes can diffuse to the polymer layer and degrade the same.
2. Sensor according to (1) characterized in that the colour is generated via pigments or dyes.
3. Sensor according to (1) characterized in that PLA, PLGA, PHB and Polyvinylcaprolactame or a different biodegradable polymer is used as polymer.
4. Sensor according to (1)-(3) characterized in that a bifunctional radical cross-linking agent is used as cross-linking agent.
5. Sensor according to (1)-(4) characterized in that the sensing layer consists of a degradable polymer, a cross-linking agent and a dye only.
6. Sensor according to (1)-(5) wherein diisocyanate is used as cross-linking agent.
7. Sensor according to (1) characterized in that a transparent layer is applied onto the coloured layer so that enzymes can diffuse or degrade to the coloured layer.
8. Sensor according to (1) characterized in that the degradation is visible with the naked eye.
9. Sensor according to (1) characterized in that the coloured biodegradable polymer is printed onto a coloured carrier.
10. Method for preparing a sensor wherein a 50 to 500 nm thick, preferably 100 to 1000 nm thick, biodegradable and coloured, cross-linked polymer layer is applied onto a carrier in such a way that enzymes can diffuse to the polymer layer and degrade the same.
11. Method for preparing a sensor according to (10) wherein the polymer layer is applied via dip-coating.
12. Method for preparing a sensor according to (10) wherein the polymer layer is applied via printing.
13. Method for preparing a sensor according to (10)-(12) wherein the polymer layer is stabilized with the help of a chemical cross-linking agent.
14. Method for preparing a sensor according to (10)-(13) wherein the biodegradable coloured polymer is mixed with a colour-pigment or dye before it is applied according to (11)-(12).
15. Method for preparing a device according to (10)-(14) wherein the sensing layer consists of a mixture of a degradable polymer, a cross-linking agent and a dye.
16. Method for preparing a device according to (10)-(15) wherein the area being an active sensing area is totally or partially coated with a transparent polymer.
17. Use of the sensor according to (1) to (16) wherein the change of the colour of the sensor-surface via the action of enzymes is determined by the naked eye.
18. Use of a sensor according to (17) wherein the comparison to a reference allows the user to detect small changes of the colour of the sensor-surface.
19. Use of a sensor according to (17) wherein the comparison of the colour of the sensor-surface to a colour of a reference allows the user the semi-quantitative detection of the amount of enzyme or the time of incubation.
20. Use of a sensor according to (17)-(19) to analyze the freshness of foods.

Freshness sensor integrated into the packaging wherein a 50-5000 nm thick biodegradable, coloured and cross-linked polymer layer is applied onto a carrier such that spoilage-causative enzymes are able to diffuse to the polymer layer and degrade the same.

The invention is further illustrated by the following examples, which should not be construed as limiting. The contents of all references, patent applications, patents, published patent applications, tables and appendices cited throughout this application are hereby incorporated by reference.

### EXAMPLES

### Example 1

### Sensor for Detecting the Concentration of Spoilage-Causative Enzymes of Meat Juice

Meat juice develops upon aging of meat. In said meat juice, enzymes are present which are either derived from spoilage-causative microorganism such as Bacillus, Clostridiae, Proteus, Aeromonas, Acinetobacter, Serratia, Flavobacterium, Actinomyces, Moulds, etc., or derived from the meat itself as for example proteinases, lipases, esterases and hydrolases, which arise on the surface due to the age-induced lysis of cells. All these enzymes possess degrading properties.

A 23, 50 or 100 μm thick polyethylenterephtalate or polyamide carrier is drawn with 6 cm/min out of a solution of 5% (by weight) PLA (Bayer, MW 200.000 Da) and 1.75% Desmodur (Jäckle Chemie) in trifluoroethanole.

Alternatively, a 23, 50 or 100 μm thick polyethylenterephtalate or polyamide carrier is drawn with 6 cm/min out of a solution of 20% (by weight) PLA (Biomer, MW 200.000 Da) and 0.001% Desmodur (Jäckle Chemie) in trifluoroethanole.

To this solution, a dye (food colouring, acryl dyes, pigments, azo dyes, fluorescence dyes, luminescence dyes) is added which is colouring the polymer. A tension-free film with a mass thickness of 120 nm is applied onto the substrate by dip coating. Also, the polymer layer may be applied by printing or by spin-coating. The resulting sensor displays within a short time frame a change of the surface-colour or a disappearance of the colour, respectively.

In the example shown in FIG. 2, a 100 μm thick polyethylenterephtalate carrier was drawn with 6 cm/min out of a solution of 5% (by weight) PLA (Bayer, MW 200.000 Da) and 1.75% Desmodur (Jäckle Chemie) in trifluoroethanole. A green food colouring (E 140) in a concentration of 8% (weight/volume) was added to the polymer solution and a tension-free film with a thickness of 120 nm was applied onto the carrier.

FIG. 2 shows a picture of a sensor-platelet which has been incubated with different concentrations of meat juice.

### Example 2

### Sensor for Detecting the Spoilage of Fish

The sensor described in example 1 is placed as an insertion platelet between, underneath or upon the fish in the conservation packaging. Thereby, the surface of the sensor is directly exposed to the juices of the fish. Enzymes derived from haematocryal animal bodies are in general more active than those derived from endothermal animals under the storage conditions used; due to this fact, fish juices arise more quickly and at lower temperatures than meat juices. That is why fish is easier perishable. The surface-colour changes continuously within 3 days at room temperature from deep blue to milky white.

### Example 3

### Sensor for Analysing the Freshness of Several Foods or a Mixture of Several Foods

By combining several printing devices in a row, it is easily and quickly possible to apply a plurality of differently coloured degradable and cross-linked polymer mixtures by printing processes and thereby to produce multi-analyte arrays. It is thus possible to produce a sensor-platelet onto which different degradable polymers are combined such that different patterns selectively react for the spoilage of certain meat-species. Using such a multi-analyte array, it is also possible to detect the spoilage of fish in one instance, in the other instance selectively the spoilage of beef. FIG. 3 shows the schematic use of a packaging with an integrated indicator. The main focus should in this circumstance be on the design of the sensor as stripe, wherein a representative measurement of the whole meat-surface is possible by the form of said stripe. The spoilage of meat often differs locally to a large extent even on the same piece of meat.

1. A device comprising a coloured and biodegradable polymer layer positioned on a carrier layer wherein the device is configured in such a way that biomolecules capable of degrading said polymer layer are allowed to contact said polymer layer and wherein the device is configured in such a way that a degradation of said polymer layer results in a colour change visible to the human eye.

2. A device according to claim 1 wherein said carrier layer has a thickness of 10 nm to 500 μm and said coloured and biodegradable polymer layer has a thickness of 50 to 5000 nm.

3. A device according to claims 1 and 2 wherein said carrier layer is selected from the group of carriers comprising polyethylenterephtalate, polyamide, glass, polyethylene, polycarbonate, polypropylene, silicone, ceramics.

4. A device according to claims 1 to 3 wherein said carrier layer is coloured.

5. A device according to claims 3 and 4 wherein said coloured carrier layer is made of polyethylenterephtalate and has a thickness of 20 to 100 μm.

6. A device according to claim 1 wherein biomolecules capable of degrading said polymer layer are allowed to penetrate said carrier layer in order to contact said polymer layer.

7. A device according to any of claims 1 to 6 wherein said coloured and biodegradable polymer layer is degradable by biomolecules comprising enzymes and/or catabolic metabolites.

8. A device according to any of claims 1 to 7 wherein said coloured and biodegradable polymer layer is selected from the group of polymers comprising PLA, PLGA, PHB and Polyvinylcaprolactame.

9. A device according to any of claims 1 to 8 wherein said coloured and biodegradable polymer layer is coloured using a dye and/or pigment selected from the group comprising food colouring, acryl-dyes, azo-dyes, fluorescence-dyes and luminescence-dyes.

10. A device according to claims 7 to 9 wherein said coloured and biodegradable polymer layer has a thickness of 100 to 1000 nm.

11. A device according to claims 7 to 9 wherein said coloured and biodegradable polymer layer additionally comprises a cross-linking agent to obtain a certain degree of cross-linking in said polymer layer wherein said cross-linking agent is selected from the group of bifunctional radical cross-linking agents.

12. A device according to any of claims 1 to 11 wherein the device also comprises a reference device.

13. A method for preparing a device according to any of claims 1 to 12 wherein said method comprises the steps of
a) Providing a carrier layer
b) Applying a coloured and biodegradable polymer layer onto said carrier layer.

14. A method according to claim 13 wherein in step a) a carrier layer is provided which is coloured in a different colour than said polymer layer.

15. A method according to claim 13 wherein the polymer layer of step b) has been coloured by mixing it with a dye and/or pigment before applying it onto said carrier layer.

16. A method according to claim 13 wherein the polymer layer is applied by dip coating or film-printing.

17. A method for analyzing the age and/or quality of a natural product comprising foods and cosmetical products which comprises the following steps:
a) Providing a device according to any of claims 1 to 12
b) Contacting said device with said natural product
c) Determining the colour of said device
d) Comparing the colour of said device with a reference device
e) Determining the age and/or quality of said natural product according to this comparison.

18. A method for analyzing the age and/or quality of a natural product according to claim 17 wherein the polymer layer of said device is being contacted in step b) directly with said natural product.

19. A method for analyzing the age and/or quality of a natural product according to claim 17 wherein the carrier layer of said device is being contacted in step b) with said natural product in such a way that biomolecules are allowed to penetrate the carrier layer and contact the polymer layer.

20. The use of a device according to any of claims 1 to 12 for the analysis of the age and/or quality of a natural product comprising foods and cosmetical products.

21. The use of a device according to claim 20 for the analysis of the age and/or quality of a natural product by detecting microorganisms present in the natural product.

22. The use of a device according to claim 20 for the analysis of the age and/or quality of a natural product by detecting enzymes and/or catabolic metabolites of microorganisms and/or of the natural product via the degradation of said biodegradable polymer by said enzymes and/or catabolic metabolites.

23. The use of a device according to claim 20 for the analysis of the age and/or quality of a natural product wherein the degree of cross-linking of the polymer layer is proportional to the kinetics of degradation of said polymer layer by enzymes and/or catabolic metabolites.
